(19) United States
(12) Patent Application Publication
(10) Pub. No.: US 2004/0068282 A1
(43) Pub. Date: Apr. 8, 2004

(54) Lancet for skin prickers

(76) Inventor: Stephen Bicknell, Ilmington (GB)

Correspondence Address: YOUNG & THOMPSON, 745 SOUTH 23RD STREET 2ND FLOOR, ARLINGTON, VA, 22202

(21) Appl. No.: 10/450,377

(22) PCT Filed: Dec. 11, 2001
(86) PCT No.: PCT/GB01/05433

(30) Foreign Application Priority Data
Dec. 14, 2000 (GB) 0030489.9

(51) Int. Cl. A61B 17/14

(52) U.S. Cl. 606/181

(57) ABSTRACT

A lancet for a skin pricker has needle (3) encased, apart from its tip (4), in a plastics body (1) that tip may initially be encased in a breakaway cap (5) integrally moulded with the body (1). The needle (3) is made from a cylindrical blank reduced to a smaller diameter near one end, whose free extremity is sharpened to form the tip (4). The transitional step or shoulder (11) between different diameter portions (9, 10) is within the plastics body (1), preventing the needle (3) moving forwards relative to that body. The reduction can be achieved by centreless cylindrical grinding, rolling or turning.

This invention relates to lancets for skin prickers. These draw a small drop of blood for analysis, and they are widely used by diabetics, for example, who need to know their sugar level.

One of the intricate and complex tasks faced in manufacturing such devices is the precision machining and grinding required to form a point on the needle tip. This typically requires three flats to be ground onto the needle tip. This is exacerbated by the desire to make the cross-section of the needle tip as small as possible to achieve a sharper point and therefore give a less noticeable prick. For an existing design such as that shown in EP-B-0 858 289 the needle blanks are taped up, i.e. attached in a row between two layers of sticky tape, and then ground in several passes to produce a flat at one end of each blank. This portion of reduced cross-sectional area then has a point formed at its tip. However, while this can give a really sharp point, the needle is of small diameter and there are difficulties arising from such lack of size during the moulding processes. It would be easier to precision machine and mould around a more substantial body, especially in high volume manufacture.

According to one aspect of the present invention there is provided a lancet for a skin pricker, the lancet having a needle encased, apart from its tip, in a body of plastics material, the needle being of generally circular cross-section within the body but with a reduction in diameter before emerging at the tip.

With this construction the diameter of the main body of the needle can be sufficiently large to enhance the rigidity of the lancet main body. It can also facilitate handling of the needle blank during its manufacturing and moulding stages.

The reduction in diameter may either be by an abrupt step or by a tapered shoulder.

Often a breakaway cap will be integrally moulded with the body to encase the needle tip. This is for safety and hygiene, and the cap may also serve as the part which the user manipulates to cock the lancet firing device. When the cap is removed the step or shoulder will prevent any tendency of the needle to shift forwards and expose more tip than intended.

The larger diameter portion of the needle preferably has a diameter in the range 0.6 to 1.0 mm, and ideally a diameter of about 0.8 mm (21 gauge). The reduced diameter portion of the needle preferably has a diameter in the range 0.2 to 0.5 mm, and the optimum is likely to be in the range 0.3 to 0.4 mm.

According to another aspect of the present invention there is provided a method of producing a lancet for a skin pricker, the method comprising:

providing a generally cylindrical needle blank,

reducing the diameter of one end portion of the blank,

forming a point on the free end or tip of said one end portion, thus creating a needle,

introducing the needle into a mould, and

introducing plastics material into said mould to form a lancet body around the needle apart from its tip.

The tip may have a breakaway plastics cap moulded around it in the same operation as the moulding of the body, as mentioned above.

The reduction of said one end portion of the blank may be by centreless cylindrical grinding, rolling or turning.

Needle blanks of a diameter corresponding to that of the main body of the finished needle may be supplied to an automated machining or working device, which machines or works the blank to produce the reduced diameter portion to form the needle tip. This is achieved without the laborious process of taping up needle blanks as previously. Since the part-formed blank (prior to needle tip formation) resulting from this process is still rotationally symmetric, further handling and manufacture of the needle and the complete lancet is facilitated.

For a better understanding of the invention, one embodiment will now be described, by way of example only, with reference to the accompanying drawing, in which:—

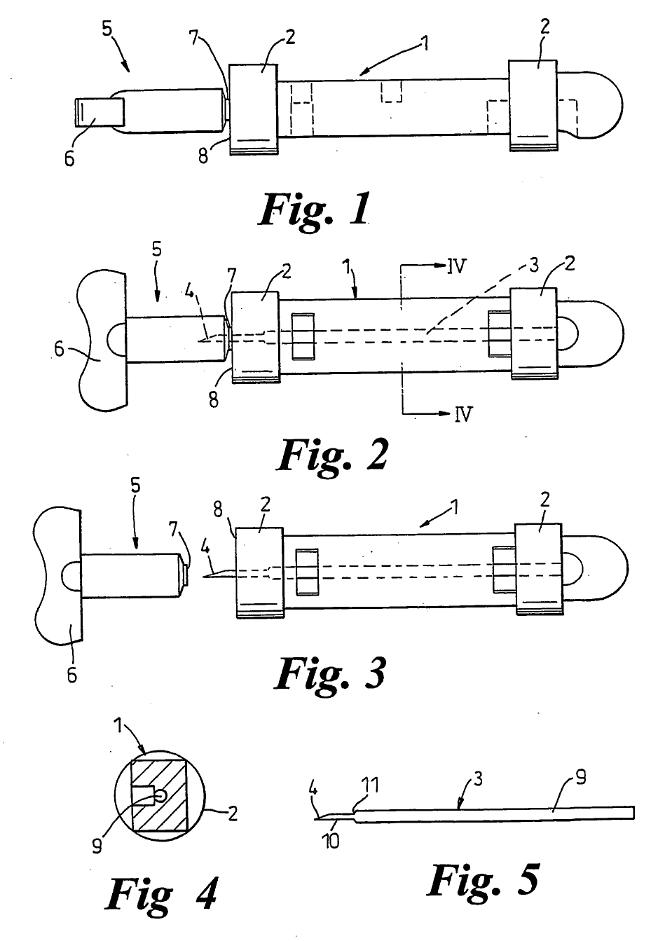

FIG. 1 is a side view of a lancet in a pre-used condition;

FIG. 2 is another side view of the lancet taken from a direction perpendicular to that of FIG. 1;

FIG. 3 is a view of the lancet similar to that of FIG. 2 but showing the breakaway cap removed,

FIG. 4 is a cross-section taken on lines iv-iv of FIG. 2, and

FIG. 5 is a side view of a needle for this lancet.

The lancet has an elongate body 1 of moulded plastics material. As is apparent in FIG. 4, the body is of generally rectangular section with two circular collars 2 at opposite ends. Coaxially encased within the body is a needle 3, shown in dotted lines in FIGS. 3 and 4 and fully in FIG. 5, and which projects in a sharp tip 4 at the forward end.

The tip 4 is initially encased in a cap 5 which is in the form of a clock key with a transverse head 6. The cap 5 is integrally moulded with the body 1 and connects to it by a neck 7 which is weak enough to be sheared off from the end surface 8 of the body by a twisting action. Such a cap may not always be provided.

The needle has a cylindrical main body portion 9 over most of its length, the remainder being a co-axial needle tip portion 10 at one end of reduced diameter produced by a process such as centreless cylindrical grinding, rolling or turning. The free extremity of this portion 10 is ground to provide the sharp tip 4. In this embodiment the outer diameter of the portion 9 is of the order of 0.8 millimetres (21 gauge) with the diameter of the needle tip portion 10 being of the order of 0.3 to 0.4 millimetres, but the invention is not limited to these dimensions or to similar proportions. It is considered reasonable for the larger diameter to be in the range 0.6 to 1.0 millimetres and the reduced one to be in the range 0.2 to 0.5 millimetres. Between the portions 9 and 10 is a shoulder 11 which is encased in the lancet body to prevent the needle from moving forwardly when the cap is removed. As shown in FIG. 5 it is an abrupt step, but it could be a tapered transition.

In one example of the manufacture of the lancet, a supply of needle blanks of the appropriate length but of constant diameter is fed to an automated centreless cylindrical grinding machine which is set up to grind the needle blanks to provide the reduced diameter tip portion 10. These rotationally symmetric intermediate blanks are then supplied to a grinding machine to form the needle point or tip 4 (typically by grinding three flats). The formed needles are then delivered to a moulding machine where they are held within the mould cavity by mould pins (not shown). The body, and cap if provided, is then moulded around the needle in plastics material. Finally, the completed lancet is ejected from the mould.

1. A lancet for a skin pricker, the lancet having a needle encased, apart from its tip, in a body of plastics material, the needle being of generally circular cross-section within the body but with a reduction in diameter before emerging at the tip.

2. A lancet as claimed in claim 1, wherein the reduction in diameter is by an abrupt step.

3. A lancet as claimed in claim 1, wherein the reduction in diameter is by a tapered shoulder.

4. A lancet as claimed in claim 1, 2 or 3, wherein a breakaway cap is integrally moulded with the body to encase the needle tip.

5. A lancet as claimed in any preceding claim, wherein the larger diameter portion of the needle has a diameter in the range 0.6 to 1.0 mm.

6. A lancet as claimed in claim 5, wherein the larger diameter portion of the needle has a diameter of 0.8 mm.

7. A lancet as claimed in any preceding claim, wherein the reduced diameter portion of the needle has a diameter in the range 0.2 to 0.5 mm.

8. A lancet as claimed in claim 7, wherein the reduced diameter portion of the needle has a diameter in the range 0.3 to 0.4 mm.

9. A method of producing a lancet for a skin pricker, the method comprising:
providing a generally cylindrical needle blank,
reducing the diameter of one end portion of the blank,
forming a point on the free end or tip of said one end portion, thus creating a needle,
introducing the needle into a mould, and
introducing plastics material into said mould to form a lancet body around the needle apart from its tip.

10. A method as claimed in claim 9, wherein the reduction of said one end portion of the blank is by cylindrical grinding.

11. A method as claimed in claim 9, wherein the reduction of said one end portion of the blank is by rolling.

12. A method as claimed in claim 9, wherein the reduction of said one end portion of the blank is by turning.
